(19) United States
(12) Patent Application Publication
(10) Pub. No.: US 2026/0218246 A1
(43) Pub. Date: Jul. 30, 2026

(54) PROCESS AND SYSTEM FOR CARBON CAPTURE BY MICROBIAL FERMENTATION

(71) Applicant: LanzaTech, Inc., Skokie, IL (US)

(72) Inventor: Zarath Morgan Summers, Prospect Heights, IL (US)

(21) Appl. No.: 19/041,210

(22) Filed: Jan. 30, 2025

(51) Int. Cl. C12P 7/04; C12M 1/00; C12M 1/107

(52) U.S. Cl. CPC C12P 7/04; C12M 21/04; C12M 47/18

(57) ABSTRACT

The disclosure is directed to a process and apparatus for capturing carbon by microbial fermentation. The disclosure relates to capturing off or waste gas stream(s) comprising CO and CO₂ produced from a cupola furnace located in a foundry. The cupola furnaces are cost-effective melting solution, but they can generate significant air emissions. The off or waste gas stream(s) from the cupola furnace is passed to gas fermentation process and converted into one or more products by microbial fermentation.

### FIELD

This disclosure relates to systems and methods for gas fermentation. In particular, the disclosure relates to the production of products from an unconventional or waste carbon source derived from an industrial source.

### BACKGROUND

The foundry industry contributes significantly to carbon emissions. Most CO and CO₂ come from manufacturing ferrous metal products such as cast iron. Despite being one of the oldest methods of converting cold scrap metals or pig irons to molten iron, the cupola furnace is still considered as cheapest and most efficient method. Most of the casting industries use cupola furnaces that emit gases namely carbon dioxide, carbon monoxide, nitrogen dioxide, sulphur dioxide, suspended particle matter, dust, and ash.

Gas fermentation has emerged as an advantageous platform for the biological fixation of carbon and transformation or utilization of carbon-rich gases such as carbon dioxide, carbon monoxide, or methane by C1-fixing microorganisms into a wide range of products such as fuel, protein, and valuable chemical compounds. These products can be used by industries in the chemical, petrochemical, pharmaceutical, animal feed, environmental and agricultural sectors. In particular, gas fermentation technology can utilize a wide range of feedstocks including gasified organic matter (for example, municipal solid waste or agricultural waste) or industrial waste gases (for example, from steel mills or foundry industry) to produce ethanol, acetate, and a variety of other products.

The present disclosure discloses system(s) and/or method(s) that provide the public with new gas fermentation methods for improved and/or increased production of a variety of useful products from carbon capture and utilization.

### SUMMARY

The disclosure provides a process of capturing carbon by microbial fermentation. The process comprises receiving off or waste gas stream(s) comprising CO and CO₂ from a cupola furnace located in a foundry; passing the off or waste gas stream(s) to at least one gas fermentation unit containing a culture of one or more microorganisms; and fermenting the culture in the gas fermentation unit to produce fermentation broth comprising one or more first product.

In an embodiment, the cupola furnace melts materials selected from one or more of cast iron, Ni-resist iron, bronzes, and any combination thereof. The off or waste gas stream(s) may also comprise NO₂ and SO₂. The process may further comprise passing the off or waste gas stream(s) to a gas treatment process before passing it to the gas fermentation unit. The gas treatment process comprises at least one process selected from catalytic reduction, adsorption, thermal oxidization, or any combination thereof. The process may further comprise passing at least a portion of the fermentation broth to a separation process for separating at least one product the fermentation broth. The separation process is selected from a fractional distillation process, an evaporation process, a pervaporation process, a gas stripping process, a phase separation process, an extractive distillation process or any combination thereof.

The one or more first product is selected from ethanol, acetate, butanol, butyrate, 2,3-butanediol, lactate, butene, butadiene, methyl ethyl ketone (2-butanone), ethylene, acetone, isopropanol, lipids, 3-hydroxypropionate (3-HP), terpenes, terpenoids, isoprene, fatty acids, 2-butanol, 1,2-propanediol, 1 propanol, chorismate-derived products, 3 hydroxybutyrate, and 1,3-butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, monoethylene glycol, 2-phenylethanol, ethylene or any combination thereof. The process may further comprise incorporating the one or more first products into one or more articles or converting the one or more first products into one or more second products. The one or more microorganism is selected from the group consisting of Clostridium autoethanogenum, Clostridium ljungdahlii, Clostridium ragsdalei, Clostridium coskatii, Wood-Ljungdahl microorganism, Cupriavidus necator, Ralstonia eutropha, Clostridium carboxidivorans, Clostridium drakei, Clostridium scatologenes, Clostridium aceticum, Clostridium formicoaceticum, Clostridium magnum, Butyribacterium methylotrophicum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kivui or any combination thereof.

The disclosure also provides an apparatus for capturing carbon by microbial fermentation, the apparatus comprising a cupola furnace in fluid communication with a first conduit and the first conduit is in fluid communication with a gas fermentation unit, the gas fermentation unit in fluid communication with a second conduit, said second conduit in fluid communication with a separation unit. The apparatus further comprising a gas treatment unit in fluid communication with the first conduit and a third conduit in fluid communication with the gas fermentation unit. The gas treatment unit is selected from a catalytic reduction unit, an adsorption unit, a thermal oxidizer unit, or any combination thereof. The separation unit is selected from a fractional distillation unit, an evaporation unit, a pervaporation unit, a gas stripping unit, a phase separation unit, an extractive fermentation unit, or any combination thereof.

The gas treatment unit is selected from a catalytic reduction unit, an adsorption unit, a thermal oxidizer unit, or any combination thereof. The separation unit is selected from a fractional distillation unit, an evaporation unit, a pervaporation unit, a gas stripping unit, a phase separation unit, an extractive fermentation unit, or any combination thereof.

### BRIEF DESCRIPTION OF THE DRAWINGS

The figures have been simplified by the deletion of a large number of apparatuses customarily employed in a process of this nature, such as vessel internals, temperature and pressure control systems, flow control valves, recycle pumps, and the like. which are not specifically required to illustrate the performance of the invention. Furthermore, the illustration of the process of this invention in the embodiment of a specific drawing is not intended to limit the disclosure to specific embodiments. Some embodiments may be described by reference to the process configuration shown in the figure, which relates to both apparatus and processes to carry out the disclosure. Any reference to a process step includes reference to an apparatus unit or equipment that is suitable to carry out the step, and vice-versa.

FIG. 1 is a schematic flow diagram showing the process and apparatus for capturing carbon by microbial fermentation, in accordance with one embodiment of the disclosure.

### DETAILED DESCRIPTION

The disclosure provides a process and apparatus for capturing carbon from a cupola furnace by microbial fermentation. The cupola furnace is a device for melting one or more materials selected from iron, Ni-resist iron, bronzes, and any combination thereof.

The gas stream(s) flowing from the cupola furnace into a gas fermentation bioreactor may be termed as “gas fermentation feedstock” or “feedstock” and should be understood to encompass any material (solid, liquid, or gas) or stream that can provide a substrate and/or C1-carbon source to a bioreactor either directly or after processing of the feedstock. The bioreactor includes a culture of one or more C1-fixing microorganisms that have the ability to produce one or more products from a C1-carbon source. “C1” refers to a one-carbon molecule, for example, CO, CO₂, CH₄, or CH₃OH. “C1-carbon source” refers to a one-carbon molecule that serves as a partial or sole carbon source for the microorganism. For example, a C1-carbon source may comprise one or more of CO, CO₂, CH₄, CH₃OH, or CH₂O₂. In an embodiment, the C1-carbon source comprises one or both of CO and CO₂. “Substrate” refers to a carbon and/or energy source for the microorganism. Typically, the substrate is gaseous and comprises a C1-carbon source, for example, CO, CO₂, and/or CH₄. The substrate may further comprise other non-carbon components, such as H₂, nitrogen compounds and sulfur compounds. In a particular embodiment the substrate and/or C1-carbon source is obtained as a by-product of the melting process using a cupola furnace.

Gas fermenting microorganisms or C1-fixing microorganisms can utilize a wide range of feedstocks including syngas generated from gasified organic matter of any sort (i.e., municipal solid waste, industrial waste, biomass, and agricultural waste residues) or industrial off-gases (i.e., from steel mills or other processing plants). In the embodiment of a cupola furnace having a height of 11 m, a diameter of 1.45 m and a rate of melting in casting of 5-6 (MT/hr) it has been reported that when excess air enters through the charging door the exhaust comprises 4.03-4.93% CO; 5.7-8.5% CO₂ plus suspended particulate matter, NO₂ and SO₂. However, when no excess air enters the furnace, the exhaust comprises 10-12% CO and 10-15% CO₂. The impurities in the substrates from the cupola furnace can affect the downstream conversion performance of gas-fermenting microorganisms. Therefore, the gas fermentation feedstock from the cupola furnace may be treated in a gas treatment unit/process to remove impurities that are fermentation inhibitors. This unit/process is also known as gas clean-up unit/process. In an embodiment, the gas treatment unit/process may comprise a hydrolysis module, an acid gas removal module, a deoxygenation module e.g. a thermal oxidization unit, a catalytic reduction unit, an adsorption unit, or any combination thereof. In another embodiment, a single module is used for all functions to remove impurities that are fermentation inhibitors. At least one gas fermentation inhibitor present in the feedstock may be removed and/or converted by the hydrolysis function. At least one of the impurities selected from carbon dioxide (CO₂), hydrogen sulfide (H₂S), and hydrogen cyanide (HCN) may be removed and/or converted by the acid gas removal unit. At least one of the impurities selected from oxygen (O₂) and/or acetylene (C₂H₂) may be removed and/or converted by the deoxygenation unit. Suitable processes for removing gas fermentation inhibitors from gas fermentation feedstocks to provide a suitable gas for a downstream fermentation process may be found in WO 2019/157519, and U.S. Pat. No. 11,441,116.

In the embodiment where a single gas treatment module is employed, at least one gas fermentation inhibitor present in the gas fermentation feedstock may be removed and/or converted in the gas treatment unit through contact with one or more specialized catalysts. For example, a specialized catalyst may be used to reduce oxygen to less than 100 ppm, acetylene to less than 1 ppm, and hydrogen cyanide to less than 1 ppm. Examples of such specialized catalysts comprise reduced copper metal on a high surface area catalyst such as silica, alumina, titania, ceria, lanthana, silica-alumina, carbon, or many other materials known to those skilled in the art. In certain instances, one specialized catalyst may be is copper (I) supported on alumina. In certain instances, the specialized catalyst comprises sulfided copper (I) supported on alumina, such that it is tolerant to sulfur. In certain instances, the specialized catalyst comprises copper (II) supported on alumina. In certain instances, the specialized catalyst comprises sulfided copper (II) supported on alumina, such that it is tolerant to sulfur. When treating feedstock with high sulfur content, the specialized catalyst may comprise sulfided copper supported on alumina.

A “C1-fixing microorganism” is a microorganism that has the ability to produce one or more products from a C1-carbon source. Typically, the microorganism contained in the bioreactor is a C1-fixing bacterium. A “microorganism” or “biocatalyst” is a microscopic organism, especially a bacterium, archaea, virus, or fungus. The microorganism is typically a bacterium. As used herein, recitation of “microbial biomass” should be taken to encompass “bacterium.”

The microorganisms contained in the bioreactor may be modified from a naturally occurring microorganism. A “parental microorganism” is a microorganism used to generate a microorganism. The parental microorganism may be a naturally occurring microorganism, known as a wild-type microorganism or a microorganism that has been previously modified, known as a mutant or recombinant microorganism. The microorganism contained in the bioreactor may be modified to express or overexpress one or more enzymes that were not expressed or overexpressed in the parental microorganism. Similarly, the microorganism contained in the bioreactor may be modified to contain one or more genes that were not contained by the parental microorganism. The microorganism contained in the bioreactor may also be modified to not express or to express lower amounts of one or more enzymes that were expressed in the parental microorganism. In one embodiment, the parental microorganism is Clostridium autoethanogenum, Clostridium ljungdahlii, or Clostridium ragsdalei. In an embodiment, the parental microorganism is Clostridium autoethanogenum LZ1561, which was deposited on Jun. 7, 2010, with Deutsche Sammlung von Mikroorganismen und Zellkulturen GmbH (DSMZ) located at Inhoffenstraβe 7B, D-38124 Braunschweig, Germany on Jun. 7, 2010, under the terms of the Budapest Treaty and accorded accession number DSM23693. This strain is described in International Patent Publication No. WO 2012/015317.

The microorganism contained in the bioreactor may be cultured with the feedstock and produce one or more gas fermentation products. For instance, the microorganism may produce or may be engineered to produce ethanol (WO 2007/117157, U.S. Pat. No. 7,972,824), acetate (WO 2007/117157, U.S. Pat. No. 7,972,824), 1-butanol (WO 2008/115080, U.S. Pat. No. 8,293,509, WO 2012/053905, U.S. Pat. No. 9,359,611 and WO 2017/066498, U.S. Pat. No. 9,738,875), butyrate (WO 2008/115080, U.S. Pat. No. 8,293,509), 2,3-butanediol (WO 2009/151342, U.S. Pat. No. 8,658,408 and WO 2016/094334, U.S. Pat. No. 10,590,406), lactate (WO 2011/112103, U.S. Pat. No. 8,900,836), butene (WO 2012/024522, US 2012/045,807), butadiene (WO 2012/024522, US 2012/045,807), methyl ethyl ketone (2-butanone) (WO 2012/024522, US 2012/045,807 and WO 2013/185123, U.S. Pat. No. 9,890,384), ethanol which is then converted to ethylene (WO 2012/026833, US 2013/157,322), acetone (WO 2012/115527, U.S. Pat. No. 9,410,130), isopropanol (WO 2012/115527 U.S. Pat. No. 9,410,130), lipids (WO 2013/036147 U.S. Pat. No. 9,068,202), 3-hydroxypropionate (3-HP) (WO 2013/180581, U.S. Pat. No. 9,994,878), terpenes, including isoprene (WO 2013/180584, U.S. Pat. No. 10,913,958), fatty acids (WO 2013/191567 U.S. Pat. No. 9,347,076), 2-butanol (WO 2013/185123 U.S. Pat. No. 9,890,384), 1,2-propanediol (WO 2014/036152, U.S. Pat. No. 9,284,564), 1-propanol (WO 2014/0369152, U.S. Pat. No. 9,284,564), 1 hexanol (WO 2017/066498, U.S. Pat. No. 9,738,875), 1 octanol (WO 2017/066498, U.S. Pat. No. 9,738,875), chorismate-derived products (WO 2016/191625, U.S. Pat. No. 10,174,303), 3-hydroxybutyrate (WO 2017/066498, U.S. Pat. No. 9,738,875), 1,3-butanediol (WO 2017/066498, U.S. Pat. No. 9,738,875), 2-hydroxyisobutyrate or 2-hydroxyisobutyric acid (WO 2017/066498, U.S. Pat. No. 9,738,875), isobutylene (WO 2017/066498, U.S. Pat. No. 9,738,875), adipic acid (WO 2017/066498, U.S. Pat. No. 9,738,875), 1,3-hexanediol (WO 2017/066498, U.S. Pat. No. 9,738,875), 3-methyl-2-butanol (WO 2017/066498, U.S. Pat. No. 9,738,875), 2-buten-1-ol (WO 2017/066498, U.S. Pat. No. 9,738,875), isovalerate (WO 2017/066498, U.S. Pat. No. 9,738,875), isoamyl alcohol (WO 2017/066498, U.S. Pat. No. 9,738,875), and/or monoethylene glycol (WO 2019/126400, US 2019/0185,888) in addition to 2-phenylethanol (WO 2021/188190, US 2021/0292732).

The microorganism contained in the bioreactor may be engineered to produce products at a certain selectivity or at a minimum selectivity. In one embodiment, the target product accounts for at least 10% of all fermentation products produced by the microorganism, such that the microorganism has a selectivity for the target product of at least 10%. In another embodiment, the target product accounts for at least 30% of all fermentation products produced by the microorganism, such that the microorganism has a selectivity for the target product of at least 30%.

The culture is generally maintained in an aqueous culture medium that contains nutrients, vitamins, and/or minerals sufficient to permit the growth of the microorganism. The aqueous culture medium may be an anaerobic microbial growth medium. The culture/fermentation should desirably be carried out under appropriate conditions for the production of the target product. Typically, the culture/fermentation is performed under anaerobic conditions. Reaction conditions to consider include pressure or partial pressure, temperature, gas flow rate, liquid flow rate, media pH, media redox potential, agitation rate when employing a continuous stirred tank reactor, inoculum level, maximum gas substrate concentrations so that gas in the liquid phase does not become limiting, and maximum product concentrations to avoid product inhibition. In particular, the rate of introduction of the substrate may be controlled so that the concentration of gas in the liquid phase does not become limiting, since products may be consumed by the culture under gas-limited conditions.

Operating a bioreactor at elevated pressures allows for an increased rate of gas mass transfer from the gas phase to the liquid phase. Accordingly, the culture/fermentation may be conducted at pressures higher than atmospheric pressure. Also, since a given gas conversion rate is, in part, a function of the substrate retention time and retention time dictates the required volume of a bioreactor, the use of pressurized systems can reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used.

The bioreactor includes a fermentation device consisting of one or more units and/or towers or piping arrangements. The bioreactor may be a continuous stirred tank reactor (CSTR), immobilized cell recycles (ICR), trickle bed reactor (TBR), bubble column, gas lift fermenter, static mixer, a circulated loop reactor, a membrane reactor, such as a hollow fibre membrane bioreactor (HFM BR) or other unit or other devices suitable for gas-liquid contact. The reactor may be adapted to receive a gaseous substrate comprising CO and/or CO₂, or H₂ or mixtures thereof. The reactor may comprise multiple reactors or stages, either in parallel or in series. For example, the reactor may comprise a first growth reactor in which the bacteria are cultured and a second fermentation reactor, to which fermentation broth from the growth reactor may be fed and in which most of the fermentation products may be produced. The fermentation process may be described as either “batch” or “continuous.” “Batch fermentation” is used to describe a fermentation process where the bioreactor is filled with raw material, i.e., the carbon source, along with microorganisms, where the products remain in the bioreactor until fermentation is completed. In a “batch” process, after fermentation is completed, the products are extracted, and the bioreactor is cleaned before the next “batch” is started. “Continuous fermentation” is used to describe a fermentation process where the fermentation process is extended for longer periods of time, and product and/or metabolite is removed during fermentation.

The fermentation broth generated from the bioreactor encompasses a mixture of components including the nutrient media, the culture of one or more microorganisms, and one or more products. “Nutrient media” or “nutrient medium” is used to describe culture growth media. Generally, this term refers to a media containing nutrients and other components appropriate for the growth of the microbial culture. The term “nutrient” includes any substance that may be utilized in a metabolic pathway of a microorganism. Exemplary nutrients include potassium, B vitamins, trace metals, and amino acids.

In an embodiment, a pump or a compressor may be provided upstream of the bioreactor so that the pressure within the bioreactor is increased. A pump or compressor may be provided to facilitate the delivery of the streams to particular stages. Furthermore, a compressor can be used to increase the pressure of gas provided to one or more stages, for example, the bioreactor. The pressure within a bioreactor can affect the solubility of gaseous substrate in the nutrient media thus increasing the efficiency of the fermentation reaction performed therein. Thus, the pressure can be adjusted to improve the efficiency of the fermentation. While the disclosure broadly describes any type of stream that may be moved through or around the system(s) by any known transfer means, in certain embodiments, the substrate and/or exhaust streams are gaseous.

The fermentation broth generated from the bioreactor may comprise waste gases generated as byproducts in gas fermentation, inert gases, and/or unused substrate gas, which may need to be removed from the broth. Typical gasses to be separated from a gas fermentation broth may include CO₂, CO, and/or H₂. The broth effluent further contains microbial biomass and liquid nutrient solution, in addition to the target product or products, and other metabolites or co-products. Waste gases not removed from the gas fermentation broth are recycled to the bioreactor with the recycled broth. Recycled inert gases and/or CO₂ dilute or restrict the amount of new gas substrate that is capable of being absorbed into the recycled broth; such new gas substrate is needed to be available for fermentation by the microbial biomass. Further, to maximize fermentation by the microbial biomass, smaller substrate bubbles should be generated by the reactor system. Smaller bubbles may be created by increasing system superficial gas velocities, see U.S. application Ser. No. 17/453,476 filed Nov. 3, 2021. Residual inert gasses and/or CO₂ not removed from the system and instead recycled in the broth may expand within the reactor restricting superficial gas velocities and limiting the creation of smaller bubbles such as fine bubbles. Therefore, at least a portion of the fermentation broth is passed to a separator. The separator operates to separate a gas stream from the fermentation broth.

In general, products may be separated from the fermentation broth using any method or combination of methods known in the art, including, for example, fractional distillation, evaporation, pervaporation, gas stripping, phase separation, extractive separation, including, for example, liquid-liquid extraction. For example, alcohols and/or ketones such as acetone may be recovered by distillation. Acids may be recovered, for example, by adsorption on activated charcoal.

A bleed stream operates to remove and prevent a small portion of the broth from being recycled to the bioreactor. However, a drawback of a bleed stream is that all components of the broth are present in the bleed stream, not just the components desired to be removed. For example, microbial biomass and nutrients in the bleed stream are removed from being recycled to the bioreactor in addition to co-products. Thus, the amount of biocatalyst and nutrients in the bioreactor would be less and may need to be replenished.

In another embodiment, substantially all of the microbial biomass produced by the fermentation process is recycled to the fermentation process after product recovery, treated by a wastewater treatment unit, and/or sent to a gasification process to produce substrate and/or C1-carbon source. In certain instances, the gasification process receives at least 20%, at least 30%, at least 40%, at least 50%, at least 60%, at least 70%, at least 80%, at least 90%, or substantially all of the microbial biomass from the fermentation process.

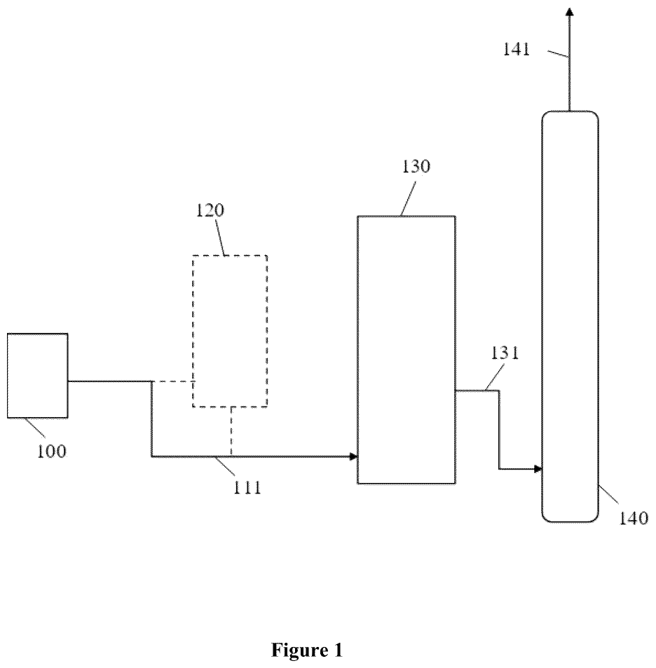

For a better understanding of the present disclosure, reference is made to the following examples and figures. Some embodiments may be described by reference to the process configurations shown in FIG. 1, which relates to both apparatus and process. Any reference to a process step includes reference to an apparatus unit or equipment that is suitable to carry out the step, and vice-versa. The following detailed description is merely exemplary and is not intended to limit the application and uses of the embodiment described.

In FIG. 1, off or waste gas stream(s) comprising CO and CO₂ is produced from a cupola furnace 100 located in a foundry. Cast iron is generally produced in a cupola furnace. Cupola furnaces can be used to melt materials selected from one or more of iron, Ni-resist iron, bronzes, and any combination thereof. Despite being one of the oldest methods of converting cold scrap metals or pig irons to molten iron, the cupola furnace is still considered as cheapest and most efficient method. The cupola furnace is a vertical cylindrical shaft where hot ascending gases produced by the combustion of coke at the bottom of the furnace come into contact with the descending melting stock. The furnace is charged at regular intervals with metallic charge, coke, and limestone.

A cupola furnace comprises a combustion zone which is an oxidizing zone. The combustion takes place due to consumption of the oxygen (generated during an air blast) by the coke to generate heat. Since the combustion reaction is exothermic, the combustion zone is the major heat source in the cupola. The reducing zone of the cupola furnace is also known as the protective zone and is located between the upper level of the combustion zone and the upper level of the coke bed. The CO₂ flowing upward through this zone reacts with hot coke and reduces to CO. The layer of metal charge above the reducing zone (i.e., the layer of the coke bed) is known as the melting zone of the cupola. The metal charge starts melting in this zone and trickles down through the coke bed and is collected in the well. The solid metal charge in the molten state picks up sufficient carbon in this zone. Preheating zone starts from the upper end of the melting zone and continues up to the bottom level of the charging door. Preheating zone includes alternate layers of coke bed, flux, and metal charge. This zone preheats the charge from room temperature to about 1090° C. before entering the metal charge into the melting zone. The hot gases rise upwards from the combustion zone and the reducing zone provides heat to the charge before exiting the furnace. Empty portion of the cupola above the preheating zone is known as the stack zone. It provides the passage for hot gases to go to the atmosphere or downstream process from the cupola furnace. The hot gases produced in the cupola furnace which leave from the stack may typically comprise carbon oxides such as carbon monoxide and carbon dioxide, sulfur oxides, and suspended particulate matter. In some situations, the hot gases produced in the cupola furnace which leave from the stack may also comprise nitrogen oxide.

Cast iron is an alloy of iron with carbon and other elements. It contains 2-6% carbon along with silicon, manganese, phosphorus, and sulfur. Cast iron is used widely in construction, machines, and decorative applications due to its low cost, durability, and ability to be cast into complex shapes. It is produced by remelting pig iron and coke in a cupola furnace. The cupola furnace uses a blast of air to remove impurities from the molten iron. The molten iron is then poured into molds to form castings.

The Ni-resist irons are a family of alloys with sufficient nickel to produce an austenitic structure which has unique and superior properties. The family may be divided into two groups. These are the standard or flake graphite alloys and the ductile or spheroidal graphite alloys. Description to make such irons is disclosed in “Engineering Properties and Applications of Ni-Resist Irons”, published by International Nickel, 5th Edition”.

Mixing the metals copper and tin creates bronze. Bronze, like other mixtures of metals, is called an alloy. Bronze is harder and stronger than copper. It also does not wear away as easily as either copper or tin. For these reasons bronze is often used to make tools and machinery. Bronze is also used to make electrical hardware, springs, fasteners, and coins. Bronze has been the most popular metal for making statues and other artistic objects since ancient times. Typically, bronze is produced by adding a small amount of melted tin to a larger amount of melted copper.

Casting and foundry are related terms, but they refer to different aspects of the process of producing metal castings. Casting is the process of creating a metal object by pouring molten metal into a mold and allowing it to cool and solidify. Castings can be used to produce a wide range of types and shapes of metal objects, from small and intricate parts to large and complex components The process of producing metal castings typically involves several steps, including making a pattern or model of the desired object, preparing a mold using sand, plaster, or ceramic, melting the metal and pouring it into the mold, allowing the metal to cool and solidify, and then removing the casting from the mold for further processing and finishing.

A foundry is a manufacturing facility that specializes in producing metal castings. Foundries use a variety of casting processes such as sand casting, die casting, investment casting, or permanent mold casting to produce a wide range of metal components in various shapes and sizes. Foundries can be categorized into different types based on their production processes and the type of metal they work with. For example, iron foundries specialize in producing iron castings, while steel foundries focus on steel castings. Non-ferrous foundries produce castings from metals like aluminum, brass, and bronze. Foundries are used to produce a wide variety of products, including machine parts, automotive components, pipes, valves, tools, art sculptures, and decorative objects. Foundries play an essential role in modern manufacturing, as they provide a cost-effective means of producing complex metal shapes in large quantities.

Cupola furnaces are commonly used in foundries for melting ferrous and non-ferrous metals. While they are an efficient and cost-effective melting solution, they can generate significant pollution, primarily in the form of air emissions. The main types of pollutants associated with cupola furnaces comprise suspended particulate matter (SPM) which includes dust, metal oxides, and other fine particles that can be harmful when inhaled. Suspended particulate matter can result from the combustion of coke and the melting of metal and slag. Carbon monoxide (CO) may be produced by the incomplete combustion of carbon in the coke used as fuel. CO is also formed in the reducing zone (above the combustion zone) of the cupola furnace where the upward flowing CO₂ reacts with hot coke and reduces the CO₂ to CO. Although in the melting zone CO reacts with iron to produce Fe₃C and CO₂, this reaction also may not go to completion or there may be excess CO resulting in CO emissions. Sulfur dioxide (SO₂) may also generate from sulfur in the coke and the metal charge. Volatile organic compounds (VOCs) can be emitted during the breakdown of organic materials in the charge. Dioxins and furans are toxic compounds that can be produced during the furnace operation, especially when burning scrap metal containing chlorinated plastics or other materials. A typical composition of the exhaust from a cupola furnace is presented above. The typical exhaust from the cupola furnace comprises majority of carbon oxides. The present disclosure provides a solution to handle to exhaust gases from the cupola furnace. In accordance with the present disclosure, the cupola furnace may be in fluid communication with the gas fermentation process to provide the exhaust gases from the cupola furnace as a feed to the gas fermentation process to produce ethanol, acetate, and a variety of other products.

Referring back to FIG. 1, the off or waste gas stream(s) from the cupola furnace 100 are passed to at least one gas fermentation unit 130. The cupola furnace 100 is in fluid communication with a first conduit 111 and the first conduit 111 is in fluid communication with a gas fermentation unit 130. In an embodiment, the off or waste gas stream(s) are passed to at least one gas fermentation unit 130 via first conduit 111. The at least one gas fermentation unit 130 comprises a culture of one or more microorganisms capable of producing a fermentation broth comprising a gas fermentation product from the off or waste gas stream(s). The gas fermentation unit 130 is in fluid communication with a second conduit 131 which is in fluid communication with a separation unit 140. The separation unit 140 may be selected from a fractional distillation unit, an evaporation unit, a pervaporation unit, a gas stripping unit, a phase separation unit, an extractive fermentation unit, or any combination thereof.

The one or more microorganism is selected from the group consisting of Clostridium autoethanogenum, Clostridium ljungdahlii, Clostridium ragsdalei, Clostridium coskatii, Wood-Ljungdahl microorganism, Cupriavidus necator, Ralstonia eutropha Clostridium carboxidivorans, Clostridium drakei, Clostridium scatologenes, Clostridium aceticum, Clostridium formicoaceticum, Clostridium magnum, Clostridium coskatii, Butyribacterium methylotrophicum, Butyribacterium limosum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kivui or any combination thereof.

In an alternate embodiment the off or waste gas stream(s) from the cupola furnace may also comprise other gases like NO₂ and SO₂. In this embodiment, the cupola furnace 100 is in fluid communication with the first conduit 111 which is in fluid communication with a gas treatment unit 120. The gas treatment unit 120 is in fluid communication with the second conduit 111 which is in fluid communication with the gas fermentation unit 130. The gas treatment unit 120 may be selected from a hydrolysis module, an acid gas removal module, a deoxygenation module, a catalytic reduction unit, an adsorption unit, a thermal oxidizer unit, or any combination thereof.

The gas fermentation unit contains the gas fermentation product and is in fluid communication with a second conduit 131. The second conduit 131 is adapted to receive the fermentation broth from the gas fermentation unit and transport it to the separation unit 140 where the gas fermentation product can be isolated, separated, processed, or any combination thereof. In an embodiment, the fermentation broth comprising one or more first products may be separated in the separation unit 140 to obtain one or more first products via conduit 141. An example of a suitable vacuum distillation unit and operation is found in U.S. Pat. Nos. 10,610,802 and 11,471,786. These separation units carry out the respective separation processes to isolate or separate the desired product.

One or more first product is at least one selected from ethanol, acetate, butanol, butyrate, 2,3-butanediol, lactate, butene, butadiene, methyl ethyl ketone (2-butanone), ethylene, acetone, isopropanol, lipids, 3-hydroxypropionate (3-HP), terpenes, terpenoids, isoprene, fatty acids, 2-butanol, 1,2-propanediol, 1 propanol, chorismate-derived products, 3 hydroxybutyrate, and 1,3-butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, monoethylene glycol, 2-phenylethanol, ethylene, single cell protein or a combination thereof. In an embodiment, the one or more first products may be incorporated into one or more articles. In another embodiment, the one or more first products may be converted into one or more second products. In one embodiment, one or more second-stage products include alcohols, acids (ex., fatty acids and omega 3 fatty acids), biofuels, alternative proteins, food products, food additives and ingredients, industrial enzymes, vitamins and nutritional supplements, antibiotics and pharmaceuticals, bioplastics, textiles and fibers, flavor and fragrance compounds, biochemicals and specialty chemicals, biogas and biomethane, biochemicals for agriculture, biopharmaceuticals, animal health products, biodegradable polymers, cellular agriculture products, amino acids, cellulosic biofuels, bio-based chemical intermediates, feed products, beverage products, medicinal products, silk proteins, cultured protein, chemicals or a combination thereof.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavor in any country.

The use of the terms “a” and “an” and “the” and similar terms are to be construed to cover both the singular and the plural, unless otherwise indicated herein or clearly contradicted by context. The terms “comprising,” “having,” “including,” and “containing” are to be construed as open-ended terms unless otherwise noted. The use of the alternative, such as the term “or,” should be understood to mean either one, both, or any combination thereof of the alternatives. As used herein, the term “about” means ±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EMBODIMENTS OF THE DISCLOSURE

Embodiment 1. A process of capturing carbon by microbial fermentation, the process comprising: receiving off or waste gas stream(s) comprising CO and CO₂ from a cupola furnace located in a foundry; passing the off or waste gas stream(s) to at least one gas fermentation unit containing a culture of one or more microorganisms; and fermenting the culture in the gas fermentation unit to produce a fermentation broth comprising one or more first product.

Embodiment 2. The process of embodiment 1 wherein the cupola furnace melts materials selected from one or more of cast iron, Ni-resist iron, bronzes, and any combination thereof.

Embodiment 3. The process of any of embodiments 1 to 2 wherein the off or waste gas stream(s) further comprises NO₂ and SO₂.

Embodiment 4. The process of any of embodiments 1 to 3 further comprising passing the off or waste gas stream(s) to a gas treatment process before passing it to the gas fermentation unit.

Embodiment 5. The process of any of embodiments 1 to 4 wherein the gas treatment process comprises at least one process selected from catalytic reduction, adsorption, thermal oxidization, or a combination thereof.

Embodiment 6. The process of any of embodiments 1 to 5 further comprising passing at least a portion of the fermentation broth to a separation unit for separating at least one product from the fermentation broth.

Embodiment 7. The process of any of embodiments 1 to 6 wherein the separation process is selected from a fractional distillation process, an evaporation process, a pervaporation process, a gas stripping process, a phase separation process, an extractive distillation process or any combination thereof.

Embodiment 8. The process of any of embodiments 1 to 7, wherein the one or more first product is at least one selected from ethanol, acetate, butanol, butyrate, 2,3-butanediol, lactate, butene, butadiene, methyl ethyl ketone (2-butanone), ethylene, acetone, isopropanol, lipids, 3-hydroxypropionate (3-HP), terpenes, terpenoids, isoprene, fatty acids, 2-butanol, 1,2-propanediol, 1 propanol, chorismate-derived products, 3 hydroxybutyrate, and 1,3-butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, monoethylene glycol, 2-phenylethanol, ethylene, single cell protein or a combination thereof.

Embodiment 9. The process of any of embodiments 1 to 8 further comprises incorporating the one or more first products into one or more articles or converting the one or more first products into one or more second products.

Embodiment 10. The process of any of embodiments 1 to 9 the one or more microorganism is selected from the group consisting of Clostridium autoethanogenum, Clostridium ljungdahlii, Clostridium ragsdalei, Clostridium coskatii, Wood-Ljungdahl microorganism, Cupriavidus necator, Ralstonia eutropha Clostridium carboxidivorans, Clostridium drakei, Clostridium scatologenes, Clostridium aceticum, Clostridium formicoaceticum, Clostridium magnum, Clostridium coskatii, Butyribacterium methylotrophicum, Butyribacterium limosum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kivui or any combination thereof.

Embodiment 11. An apparatus for capturing carbon by microbial fermentation, the apparatus comprising: a cupola furnace in fluid communication with a first conduit and the first conduit is in fluid communication with a gas fermentation unit; a gas fermentation unit in communication with a second conduit, said second conduit in fluid communication with a separation unit.

Embodiment 12. The apparatus of any of embodiments 11 further comprising a gas treatment unit in fluid communication with the first conduit and a third conduit in fluid communication with the gas fermentation unit.

Embodiment 13. The apparatus of any of embodiments 11 and 12 wherein the gas treatment unit is selected from a catalytic reduction unit, an adsorption unit, a thermal oxidizer unit, or any combination thereof.

Embodiment 14. The apparatus of any of embodiments 11 to 13 wherein the separation unit is selected from a fractional distillation unit, an evaporation unit, a pervaporation unit, a gas stripping unit, a phase separation unit, an extractive fermentation unit, or any combination thereof.

1. A process of capturing carbon by microbial fermentation, the process comprising:
i. receiving off or waste gas stream(s) comprising CO and CO₂ from a cupola furnace located in a foundry;
ii. passing the off or waste gas stream(s) to at least one gas fermentation unit containing a culture of one or more microorganisms; and
iii. fermenting the culture in the gas fermentation unit to produce a fermentation broth comprising one or more first product.

2. The process of claim 1, wherein the cupola furnace melts materials selected from one or more of iron, Ni-resist iron, bronzes, and any combination thereof.

3. The process of claim 1, wherein the off or waste gas stream(s) further comprises NO₂ and SO₂.

4. The process of claim 1, further comprising passing the off or waste gas stream(s) to a gas treatment process before passing it to the gas fermentation unit.

5. The process of claim 4, wherein the gas treatment process comprises at least one process selected from a hydrolysis process, an acid gas removal process, a deoxygenation process, a catalytic reduction process, an adsorption process, a thermal oxidization process, or any combination thereof.

6. The process of claim 1, further comprising passing at least a portion of the fermentation broth to a separation process for separating at least one product from the fermentation broth.

7. The process of claim 6, wherein the separation process is selected from a fractional distillation process, an evaporation process, a pervaporation process, a gas stripping process, a phase separation process, an extractive distillation process or any combination thereof.

8. The process of claim 1, wherein the one or more first product is selected from ethanol, acetate, butanol, butyrate, 2,3-butanediol, lactate, butene, butadiene, methyl ethyl ketone (2-butanone), ethylene, acetone, isopropanol, lipids, 3-hydroxypropionate (3-HP), terpenes, terpenoids, isoprene, fatty acids, 2-butanol, 1,2-propanediol, 1 propanol, chorismate-derived products, 3 hydroxybutyrate, and 1,3-butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, monoethylene glycol, 2-phenylethanol, ethylene, single cell protein or any combination thereof.

9. The process of claim 1, further comprising incorporating the one or more first product into one or more article or converting the one or more first product into one or more second product.

10. The process of claim 1, wherein the one or more microorganism is selected from the group consisting of Clostridium autoethanogenum, Clostridium ljungdahlii, Clostridium ragsdalei, Clostridium coskatii, Wood-Ljungdahl microorganism, Cupriavidus necator, Ralstonia eutropha Clostridium carboxidivorans, Clostridium drakei, Clostridium scatologenes, Clostridium aceticum, Clostridium formicoaceticum, Clostridium magnum, Clostridium coskatii, Butyribacterium methylotrophicum, Butyribacterium limosum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kivui or any combination thereof.

11. An apparatus for capturing carbon by microbial fermentation, the apparatus comprising:
i. a cupola furnace in fluid communication with a first conduit and the first conduit is in fluid communication with a gas fermentation unit;
ii. the gas fermentation unit in fluid communication with a second conduit, said second conduit in fluid communication with a separation unit.

12. The apparatus of claim 11, further comprising a gas treatment unit in fluid communication with the first conduit and in fluid communication with the gas fermentation unit.

13. The apparatus of claim 12, wherein the gas treatment unit is selected from a catalytic reduction unit, an adsorption unit, a thermal oxidizer unit, or any combination thereof.

14. The apparatus of claim 11, wherein the separation unit is selected from a fractional distillation unit, an evaporation unit, a pervaporation unit, a gas stripping unit, a phase separation unit, an extractive fermentation unit, or any combination thereof.
